(19) United States
(12) Patent Application Publication
(10) Pub. No.: US 2005/0027341 A1
(43) Pub. Date: Feb. 3, 2005

(54) System and method for providing a medical lead body having conductors that are wound in opposite directions

(75) Inventors: Michael P. Schrom, Wyoming Township, MN (US); Charles F. Lehman, Minneapolis, MN (US); Mark Gerald Schrom, Hugo, MN (US)

Correspondence Address: DOCKET CLERK, DM/ANSI, P.O. BOX 802432, DALLAS, TX, 75380, US

(73) Assignee: MICRONET MEDICAL, INC., White Bear Lake, MN (US)

(21) Appl. No.: 10/630,376

(22) Filed: Jul. 29, 2003

(51) Int. Cl. A61N 1/05

(52) U.S. Cl. 607/116

(57) ABSTRACT

An implantable medical lead and lead body, method of manufacturing the same, and a system and method for stimulating a portion of a body are disclosed. A lead body assembly is formed by preparing a first layer unitary body comprising a first plurality of conductors wound in a first direction. An inner layer of extrusion material is placed on the first layer unitary body. A second plurality of conductors coated with extrusion material is placed on the inner layer. The second plurality of conductors is wound in a second opposite direction. An outer layer of extrusion material is placed over the second plurality of conductors. Heat shrink tubing is placed over the assembly. The extrusion material is heated and melted and compressed around the conductors. When the heat shrink tubing is removed, the solidified extrusion material forms a protective wall that encapsulates the conductors in the lead body.

### CROSS-REFERENCE TO RELATED PATENT DOCUMENTS

The present disclosure is related to the inventions disclosed in the following U.S. patent applications:

U.S. patent application Ser. No. [Attorney Docket Number 03-002] filed concurrently herewith, entitled “System and Method for Providing A Medical Lead Body”; and

U.S. patent application Ser. No. [Attorney Docket Number 03-003] filed concurrently herewith, entitled “System and Method for Providing A Medical Lead Body Having Dual Conductor Layers.”

These patent applications are commonly owned by the assignee of the present invention. The disclosures of the related United States patent applications are incorporated herein by reference for all purposes as if fully set forth herein.

### TECHNICAL FIELD OF THE INVENTION

The present invention generally relates to medical leads and, more particularly, to a system and method for manufacturing an implantable lead that includes a lead body having a first plurality of conductors wound in a first direction and a second plurality of conductors wound in a second opposite direction.

### BACKGROUND OF THE INVENTION

Electrical signals may be used in a variety of medical applications to provide electrical stimulation to various parts of the body of a patient. For example, electrical signals may be used to modulate the amount of pain perceived by a patient by electrically stimulating a site near one or more nerves of the patient. A source of electrical signals may be implanted within the body of a patient. Electrical signals are conducted from the source of electrical signals to the stimulation site of the patient through a lead implanted within the body of the patient.

A lead generally includes a thin, flexible, lead body that contains electrically conducting conductors (e.g., wires) that extend from a first end of the lead (the proximal end) to a second end of the lead (the distal end). The lead body includes insulating material for covering and electrically insulating the electrically conducting conductors. The proximal end of the lead further includes an electrical contact that may be coupled to a source of electrical signals and the distal end of the lead includes an electrode that may be placed at the stimulation site within the body of the patient.

The use of mechanical combs can also sometimes damage the conductors. Prior art manufacturing methods can also result in a lead body that has variable (non-uniform) conductor pitches for the conductors in the lead body. Prior art manufacturing methods can also result in a lead body that has variable (non-uniform) wall thicknesses. Prior art manufacturing methods also can result in the creation of lead bodies that have relatively large diameters.

Larger electrode-carrying catheters in the prior art (such as those used in cardiology applications) may utilize electrically conducting wires that are spirally wound around a cylindrically shaped wire core. For example, U.S. Pat. No. 5,417,208 issued to Winkler describes an electrode-carrying catheter that comprises insulated wires (or non-insulated wires) that are spirally wound under hand tension around a cylindrically symmetrical wire core. The wires are embedded in a soft plastic covering (such as polyurethane having a durometer hardness of 80A available under the trade name Tecoflex) over-extruded over the wire core. The wires are embedded in the plastic covering to preclude accidental movement of the wires with respect to the wire core. Subsequently, an insulating layer of plastic is over-extruded over the soft core covering layer. This insulating layer forms a hard outer layer.

However, as more electrodes are added to the electrode-carrying catheters or leads, the more conductors are needed within the leads to connect the electrodes to the power source. The current art solves this problem by simply placing more wires, either along the length of or uniformly coiled around the lumen of the lead. This can cause the lead thickness to increase to a point that is unacceptable for its intended use in the human body.

There is a need in the art for an improved system, lead and method for manufacturing a lead body. In particular, there is a need in the art for a system, lead and method for manufacturing a lead body that is a minimal diameter from the present art, capable of protecting and accurately placing electrically conducting conductors within the lead body during the manufacturing process, and can transfer torque from the proximal end to the distal end of the lead.

### SUMMARY OF THE INVENTION

The present invention is directed to a system and method for manufacturing a lead that includes a first layer that comprises a first plurality of conductors wound in a first direction and a second layer that comprises a second plurality of conductors wound in a second opposite direction.

In one advantageous embodiment, the present invention comprises a lead for implantation into a human body. The lead comprises a lead body assembly that comprises (1) a wall having an inner portion that defines a lumen, (2) a first layer having at least one conductor wound around the lumen in a first direction, and (3) a second layer having at least one conductor wound around the lumen in a second direction and interior to the outside of the wall. The lead further comprises (4) at least one electrode located at a distal end of the lead wherein the at least one electrode is connected to at least one conductor of the first plurality of conductors, (5) at least one electrode located at a distal end of the lead wherein the at least one electrode is connected to at least one conductor of the second plurality of conductors, (6) at least one connector located at a proximal end of the lead wherein the at least one connector is connected to at least one conductor of the first plurality of conductors, and (7) at least one connector located at a proximal end of the lead wherein the at least one connector is connected to at least one conductor of the second plurality of conductors.

In another advantageous embodiment, the first layer of the lead body assembly is a first unitary body and the second layer of the lead body assembly is a second unitary body.

In another advantageous embodiment, the present invention comprises a method for manufacturing a lead. The method comprises the steps of (1) placing on a mandrel, a first layer that comprises at least one conductor of a first plurality of conductors wherein the at least one conductor of the first plurality of conductors is spirally wound in a first direction, (2) placing a second layer on the first layer, wherein the second layer comprises at least one conductor of a second plurality of conductors wherein in the at least one conductor of the second plurality of conductors is spirally wound in a second direction, (3) forming a lead body assembly that comprises the first layer and the second layer, (4) attaching at least one electrode located at a distal end of the lead body assembly to at least one conductor of the first plurality of conductors, (5) attaching at least one connector located at a proximal end of the lead body assembly to at least one conductor of said first plurality of conductors, (6) attaching at least one electrode located at a distal end of the lead body assembly to at least one conductor of the second plurality of conductors, (7) attaching at least one connector located at a proximal end of the lead body assembly to at least one conductor of the second plurality of conductors, and (8) removing the lead body assembly from the mandrel.

In another advantageous embodiment, the present invention comprises a system for stimulating a portion of a body wherein the system comprises a source for generating a stimulus, and a lead for receiving the stimulus from the source, wherein the lead comprises a lead body assembly that comprises (1) a wall having an inner portion that defines a lumen, (2) a first layer having at least one conductor wound around the lumen in a first direction, and (3) a second layer having at least one conductor wound around the lumen in a second direction and interior to the outside of the wall. The lead further comprises (4) at least one electrode located at a distal end of the lead wherein the at least one electrode is connected to at least one conductor of the first plurality of conductors, (5) at least one electrode located at a distal end of the lead wherein the at least one electrode is connected to at least one conductor of the second plurality of conductors, (6) at least one connector located at a proximal end of the lead wherein the at least one connector is connected to at least one conductor of the first plurality of conductors, and (7) at least one connector located at a proximal end of the lead wherein the at least one connector is connected to at least one conductor of the second plurality of conductors.

The foregoing has outlined rather broadly the features and technical advantages of the present invention so that those skilled in the art may better understand the detailed description of the invention that follows. Additional features and advantages of the invention will be described hereinafter that form the subject of the claims of the invention. Those skilled in the art should appreciate that they may readily use the conception and the specific embodiment disclosed as a basis for modifying or designing other structures for carrying out the same purposes of the present invention. Those skilled in the art should also realize that such equivalent constructions do not depart from the spirit and scope of the invention in its broadest form.

### BRIEF DESCRIPTION OF THE DRAWINGS

For a more complete understanding of the present invention, and the advantages thereof, reference is now made to the following descriptions and the accompanying drawings, wherein like numbers designate like objects, and in which:

FIG. 1 illustrates a perspective view of a lead constructed in accordance with the present invention;

FIG. 2 illustrates a lead of the present invention connected to a stimulation source that includes an implantable pulse generator (IPG);

FIG. 3 illustrates a lead of the present invention connected to a stimulation source that includes a radio frequency receiver;

FIG. 4 illustrates a cross sectional view of an advantageous embodiment of a first layer unitary body assembly comprising an inner layer of extrusion material, a first plurality of conductors coated with a layer of extrusion material wrapped around the inner layer in a first direction, and an outer layer of extrusion material;

FIG. 5 illustrates a cross sectional view of an advantageous embodiment of a first layer unitary body formed by subjecting the first layer unitary body assembly shown in FIG. 4 to melting and compression;

FIG. 6 illustrates a cross sectional view of a first embodiment of a lead body assembly of the present invention comprising a first layer unitary body as shown in FIG. 5 and a second layer comprising an inner layer of extrusion material, a second plurality of conductors coated with a layer of extrusion material wherein the second plurality of conductors are wrapped around the inner layer of extrusion material in a second direction that is opposite to the direction of the first plurality of conductors in the first layer unitary body, and an outer layer of extrusion material;

FIG. 7 illustrates a cross sectional view of a first embodiment of the lead body of the present invention formed by subjecting the lead body assembly shown in FIG. 6 to melting and compression;

FIG. 8 illustrates a cross sectional view of a second embodiment of a lead body assembly of the present invention comprising a first layer unitary body as shown in FIG. 5 and a second layer comprising a second plurality of conductors coated with a layer of extrusion material wherein the second plurality of conductors are wrapped around the first layer unitary body in a second direction that is opposite to the direction of the first plurality of conductors in the first layer unitary body, and an outer layer of extrusion material;

FIG. 9 illustrates a cross sectional view of a second embodiment of the lead body of the present invention formed by subjecting the lead body assembly shown in FIG. 8 to melting and compression;

FIG. 10 illustrates a cross sectional view of a third embodiment of a lead body assembly of the present invention comprising a first layer unitary body as shown in FIG. 5 and a second layer comprising an inner layer of extrusion material and a second plurality of conductors coated with a layer of extrusion material wherein the second plurality of conductors are wrapped around the inner layer of extrusion material in a second direction that is opposite to the direction of the first plurality of conductors in the first layer unitary body;

FIG. 11 illustrates a cross sectional view of a third embodiment of the lead body of the present invention formed by subjecting the lead body assembly shown in FIG. 10 to melting and compression;

FIG. 12 illustrates a cross sectional view of a fourth embodiment of a lead body assembly of the present invention comprising a first layer unitary body as shown in FIG. 5 and a second layer comprising a second plurality of conductors coated with a layer of extrusion material wherein the second plurality of conductors are wrapped around the first layer unitary body in a second direction that is opposite to the direction of the first plurality of conductors in the first layer unitary body;

FIG. 13 illustrates a cross sectional view of a fourth embodiment of the lead body of the present invention formed by subjecting the lead body assembly shown in FIG. 12 to melting and compression;

FIG. 14 illustrates a cross sectional view of a fifth embodiment of a lead body assembly of the present invention comprising a first layer unitary body as shown in FIG. 5 and a s second layer comprising a second plurality of conductors wherein the second plurality of conductors are wrapped around the first layer unitary body in a second direction that is opposite to the direction of the first plurality of conductors in the first layer unitary body, and an outer layer of extrusion material;

FIG. 15 illustrates a cross sectional view of a fifth embodiment of the lead body of the present invention formed by subjecting the lead body assembly shown in FIG. 14 to melting and compression;

FIG. 16 is a flow diagram illustrating the steps of an advantageous embodiment of a method for making a first embodiment of the lead body of the present invention;

FIG. 17 is a flow diagram illustrating the steps of an advantageous embodiment of a method for making a second embodiment of the lead body of the present invention;

FIG. 18 is a flow diagram illustrating the steps of an advantageous embodiment of a method for making a third embodiment of the lead body of the present invention;

FIG. 19 is a flow diagram illustrating the steps of an advantageous embodiment of a method for making a fourth embodiment of the lead body of the present invention;

FIG. 20 is a flow diagram illustrating the steps of an advantageous embodiment of a method for making a fifth embodiment of the lead body of the present invention;

FIG. 21 illustrates a longitudinal cross sectional view of a first layer unitary body of the present invention showing heat shrink material attached at each end of the first layer unitary body;

FIG. 22 illustrates a longitudinal cross sectional view of one end of the lead body of the present invention showing the application of heat shrink material to the end of the lead body to separate the first and second plurality of conductors;

FIG. 23 illustrates a cross sectional view of one end of the lead body of the present invention where the lead body is covered with a portion of heat shrink material;

FIG. 24 illustrates a cross sectional view of one end of the lead body of the present invention at a point where the lead body is covered with heat shrink material and at a point where the first layer unitary body of the present invention is also covered with heat shrink material;

FIG. 25 illustrates a perspective side view of a mandrel with an exemplary conductor of a first plurality of conductors wound around the mandrel in a first direction in an inner layer of conductors and an exemplary conductor of a second plurality of conductors wound around the mandrel in a second direction in an outer layer of conductors;

FIG. 26 illustrates a perspective side view of a mandrel with an exemplary first conductor wound around the mandrel in a first direction in an inner layer and an exemplary second conductor wound around the mandrel in a second opposite direction in a second layer; and

FIG. 27 illustrates a perspective side view of a mandrel with an exemplary first plurality of conductors wound around the mandrel in a first direction in an inner layer of conductors and an exemplary second plurality of conductors wound around the mandrel in a second opposite direction in a second layer of conductors.

### DETAILED DESCRIPTION OF THE INVENTION

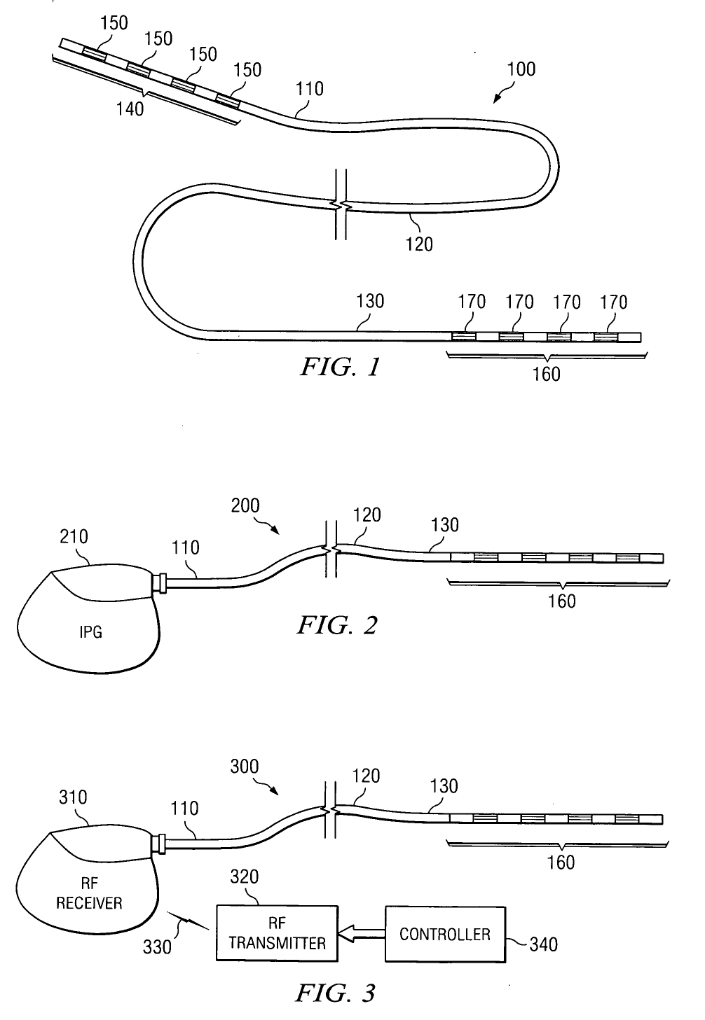

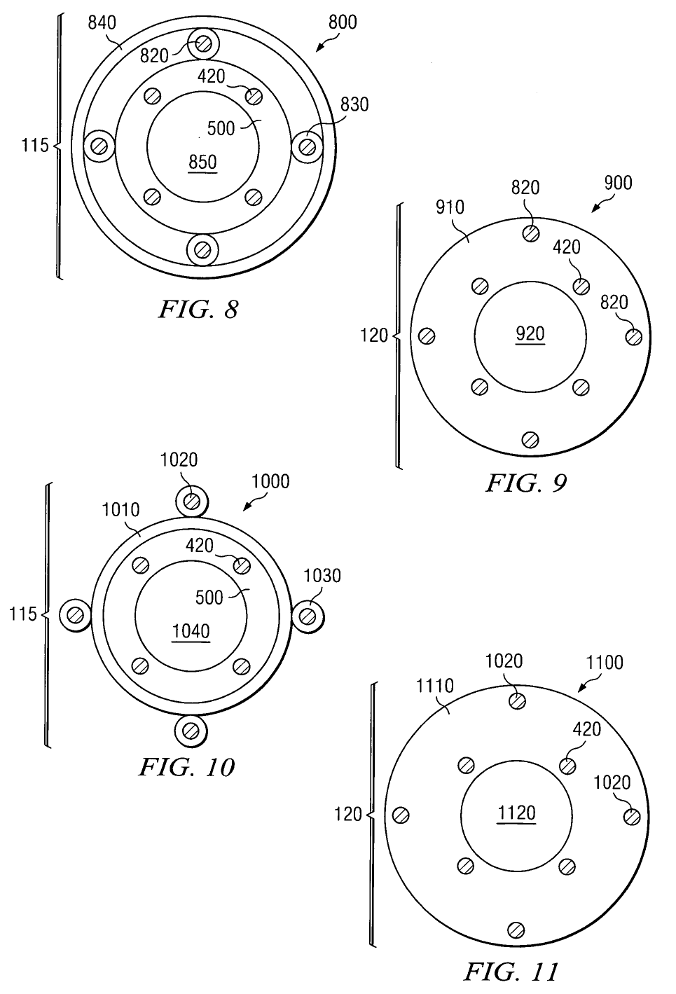

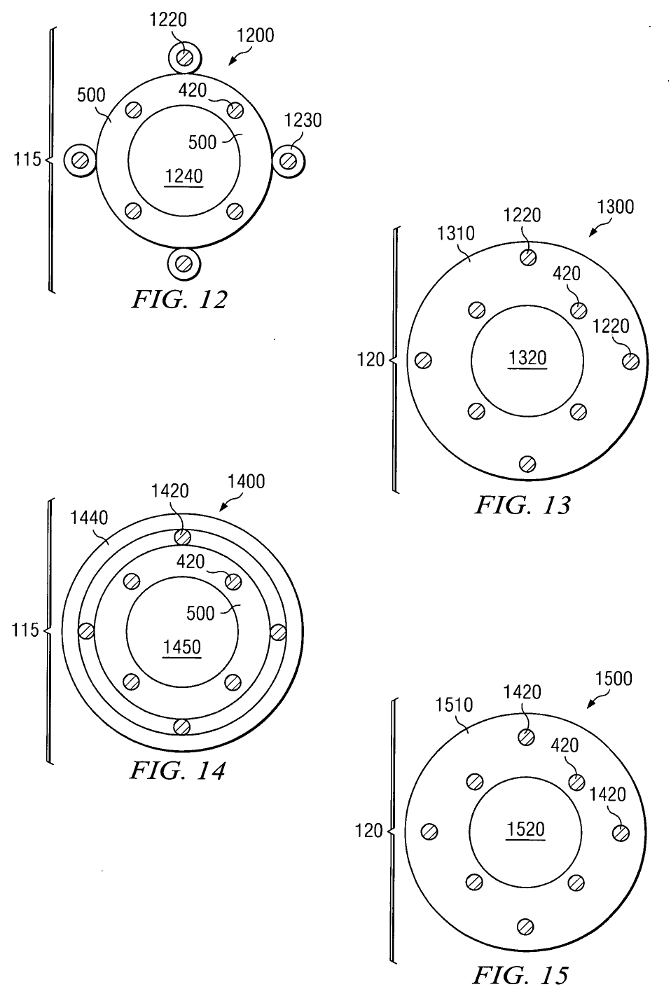

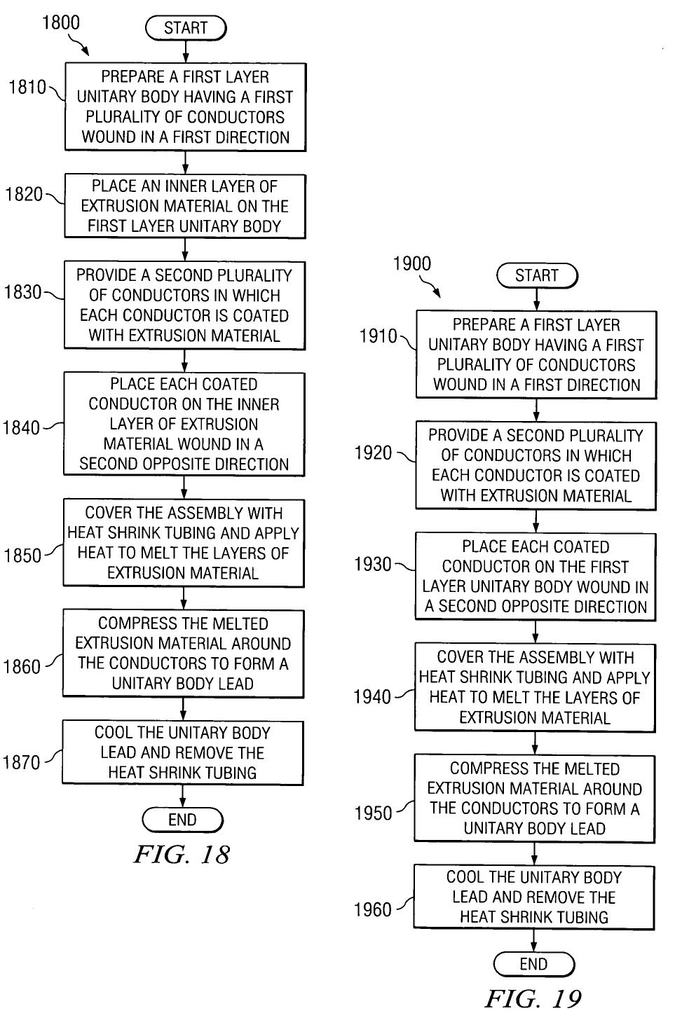

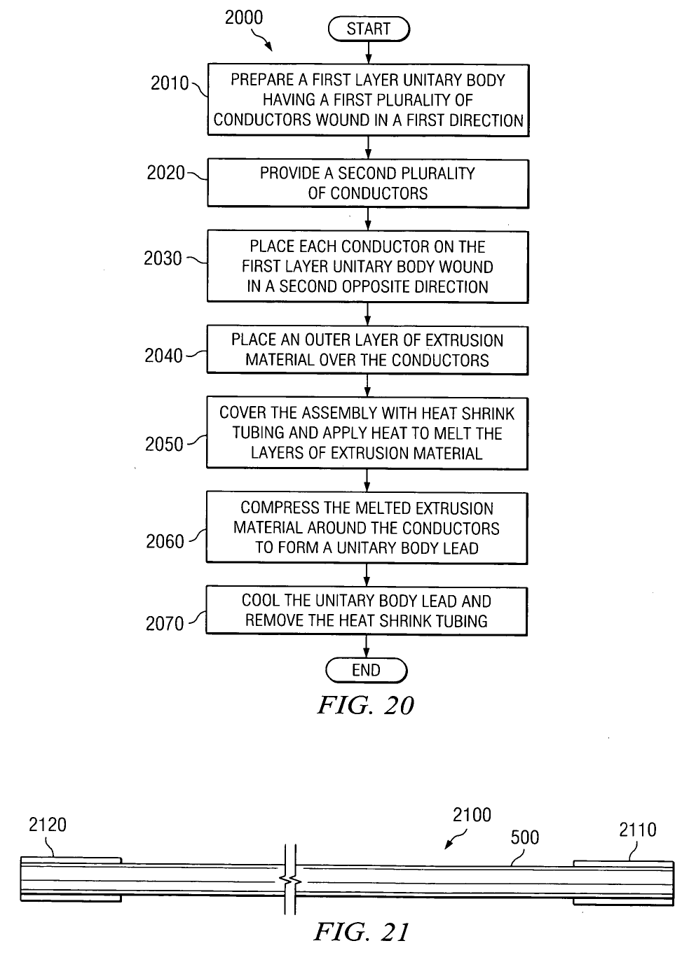

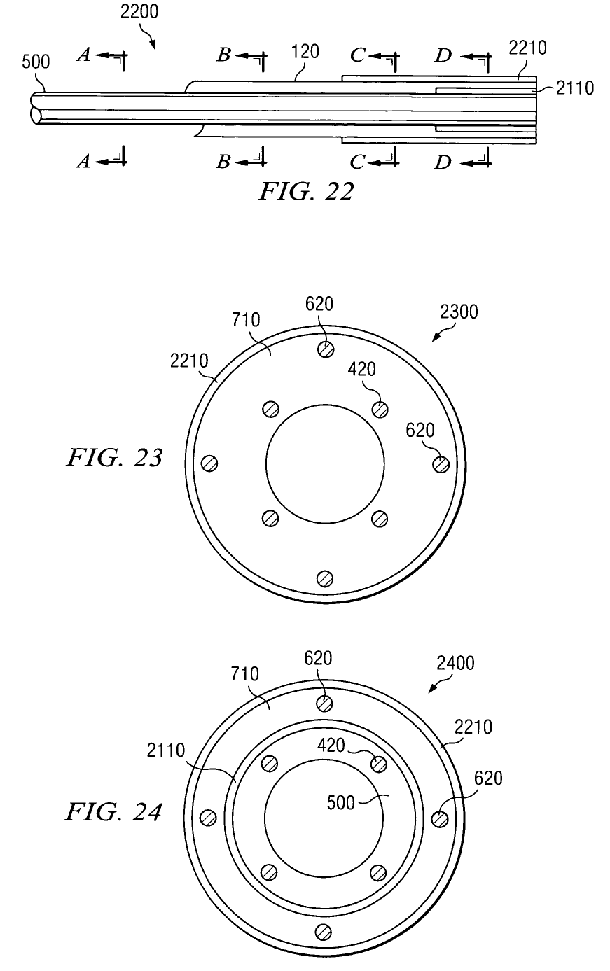

FIGS. 1 through 27, discussed below, and the various embodiments used to describe the principles of the present invention in this patent document are by way of illustration only and should not be construed in any way to limit the scope of the invention. Those skilled in the art will understand that the principles of the present invention may be implemented in any suitably modified medical lead.

U.S. patent application Ser. No. [Attorney Docket Number 03-002] filed concurrently herewith, entitled “System and Method for Providing A Medical Lead Body” fully and U.S. patent application Ser. No. [Attorney Docket Number 03-003] filed concurrently herewith, entitled “System and Method for Providing A Medical Lead Body Having Dual Conductor Layers”, which are incorporated by reference herein, fully disclose, describe and teach a system, lead and their associated manufacturing methodology. These applications are incorporated by reference here in full.

FIG. 1 illustrates an advantageous embodiment of a lead 100 of the present invention. Lead 100 includes a flexible lead body 120 having a proximal end 110 and a distal end 130. Proximal end 110 of lead body 120 is coupled to an electrical contact 140. Distal end 130 of lead body 120 is coupled to electrode 160. Electrical contact 140 includes portions of lead body 120 and a plurality of contact electrodes 150 (also sometimes referred to as ring electrodes 150). Electrode 160 includes portions of lead body 120 and a plurality of band electrodes 170 (also sometimes referred to as ring electrodes 170). Although four contact electrodes 150 and four band electrodes 170 are shown in FIG. 1, it is understood that the present invention is not limited to the use of exactly four contact electrodes 150 or four band electrodes 170.

FIG. 2 and FIG. 3 illustrate different embodiments of a system (200, 300) for generating and applying a stimulus to a tissue or to a certain location of a body. In general terms, the system (200, 300) includes a stimulation or energy source (210, 310) and a lead 100 for application of the stimulus. The lead 100 shown in FIG. 2 and in FIG. 3 is the lead of the present invention.

FIG. 2 illustrates a lead 100 of the present invention connected to a stimulation source 210. The stimulation source 210 shown in FIG. 2 includes an implantable pulse generator (IPG). As is well known in the art, an implantable pulse generator (IPG) is capable of being implanted within a body (not shown) that is to receive electrical stimulation from the stimulation source 210. An exemplary implantable pulse generator (IPG) may be one manufactured by Advanced Neuromodulation Systems, Inc., such as the Genesis® System, part numbers 3604, 3608, 3609, and 3644. Reference numeral 200 refers to the system including the lead 100 and the stimulation source 210.

Electrical contact 140 is not visible in FIG. 2 because electrical contact 140 is situated within a receptacle (not shown) of stimulation source 210. Electrical contact 140 is electrically connected to a generator (not shown) of electrical signals within stimulation source 210. Stimulation source 210 generates and sends electrical signals via lead 100 to electrode 160. Electrode 160 is located at a stimulation site (not shown) within the body that is to receive electrical stimulation from the electrical signals. A stimulation site may be, for example, adjacent to one or more nerves in the central nervous system (e.g., spinal cord). The band electrodes 170 of electrode 160 conduct electrical signals from electrode 160 to the stimulation site. Stimulation source 210 is capable of controlling the electrical signals by varying signal parameters (e.g., intensity, duration, frequency) in response to control signals that are provided to stimulation source 210.

FIG. 3 illustrates a lead 100 of the present invention connected to a stimulation source 310. The stimulation source 310 shown in FIG. 3 includes a radio frequency (RF) receiver. As is well known in the art, a stimulation source 310 comprising a radio frequency (RF) receiver is capable of being implanted within the body (not shown) that is to receive electrical stimulation from the stimulation source 310. Exemplary RF receiver 310 may be those RF receivers manufactured by Advanced Neuromodulation Systems, Inc., such as the Renew® System, part numbers 3408 and 3416. Reference numeral 300 refers to the system including the lead 100 and the stimulation source 310. System 300 may also include the optional components 320 and 340 described below.

Electrical contact 140 is not visible in FIG. 3 because electrical contact 140 is situated within a receptacle (not shown) of stimulation source 310. Electrical contact 140 is electrically connected to a generator (not shown) of electrical signals within stimulation source 310. Stimulation source 310 generates and sends electrical signals via lead 100 to electrode 160. Electrode 160 is located at a stimulation site (not shown) within the body that is to receive electrical stimulation from the electrical signals. A stimulation site may be, for example, adjacent to one or more nerves in the central nervous system (e.g., spinal cord). The band electrodes 170 of electrode 160 conduct electrical signals from electrode 160 to the stimulation site. Stimulation source 310 is capable of controlling the electrical signals by varying signal parameters (e.g., intensity, duration, frequency) in response to control signals that are provided to stimulation source 310.

As shown in FIG. 3, the radio frequency (RF) receiver within stimulation source 310 is capable of receiving radio signals from a radio frequency (RF) transmitter 320. The radio signals are represented in FIG. 3 by radio link symbol 330. Radio frequency (RF) transmitter 320 and controller 340 are located outside of the body that is to receive electrical stimulation from stimulation source 310. A user of stimulation source 310 may use controller 340 to provide the control signals for the operation of stimulation source 310. Controller 340 provides the control signals to radio frequency (RF) transmitter 320. Radio frequency (RF) transmitter 320 transmits the control signals to the radio frequency (RF) receiver in stimulation source 310. Stimulation source 310 uses the control signals to vary the signal parameters of the electrical signals that are transmitted through electrical contact 140, lead body 120, and electrode 160 to the stimulation site. Exemplary RF transmitter 320 may be those RF transmitters manufactured by Advanced Neuromodulation Systems, Inc., such as the Renew® System, part numbers 3508 and 3516.

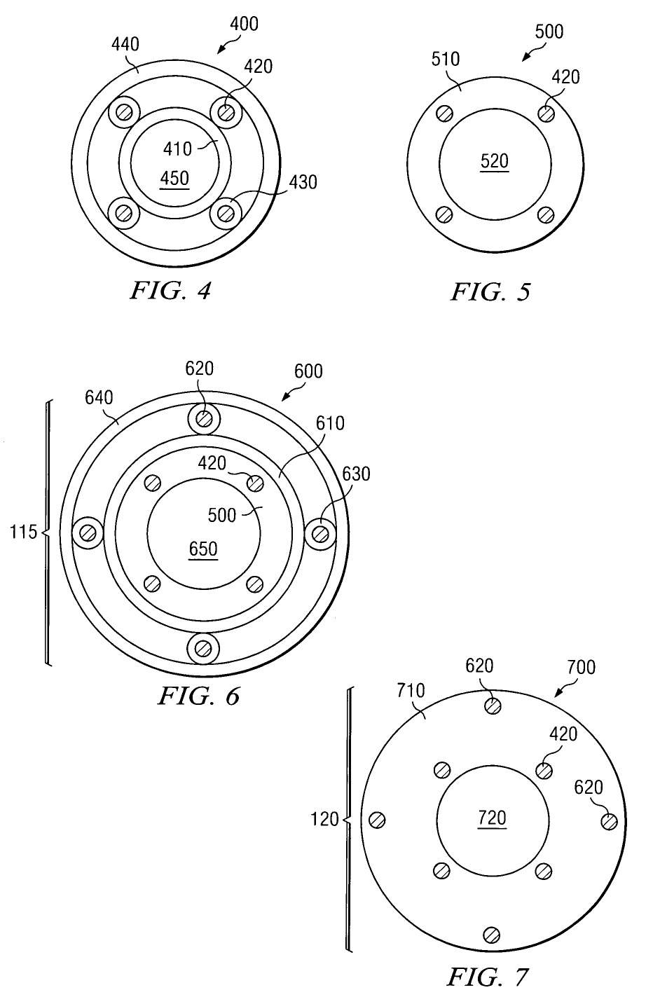

FIG. 4 illustrates a cross sectional view of an advantageous embodiment of a first layer unitary body assembly 400 of the lead body 120 of the present invention. The first layer unitary body assembly 400 of lead body 120 includes (1) an inner layer 410 of extrusion material, (2) a first plurality of conductors 420 in which each conductor 420 is coated with a layer of extrusion material 430, and (3) an outer layer 440 of extrusion material. A lumen 450 is formed by the inner wall of inner layer 410.

An advantageous embodiment of a method for making a first layer unitary body 500 of lead body 120 (shown in FIG. 5) will now be described. An inner layer 410 of extrusion material is placed on a cylindrically shaped mandrel (not shown). After the first layer unitary body assembly 400 is removed from the mandrel, the space formerly occupied by the mandrel will form lumen 450 within inner layer 410. Each conductor 420 of the first plurality of conductors 420 is coated with a layer 430 of the same extrusion material that is used to form inner layer 410. Alternatively, the extrusion material used to form layer 430 may not be the same type of extrusion material that is used to form inner layer 410. Each conductor 420 of the first plurality of conductors 420 is wrapped around (i.e., coiled around) the inner layer 410 of extrusion material (either in a clockwise direction or in a counterclockwise direction). The layer 430 of extrusion material around each conductor 420 ensures that the conductors 420 are uniformly spaced. An outer layer 440 of extrusion material is placed over the first plurality of conductors 420. The outer layer 440 of extrusion material forms an external coating over the first plurality of conductors 420 as shown in FIG. 4.

The extrusion material is formed of an insulating material typically selected based upon biocompatibility, biostability and durability for the particular application. The extrusion material may be silicone, polyurethane, polyethylene, polyimide, polyvinylchloride, PTFT, EFTE, or other suitable materials known to those skilled in the art. Alloys or blends of these materials may also be formulated to control the relative flexibility, torqueability, and pushability of the lead body 120. Depending on the particular application, the diameter of the lead body 120 may be any size, though a smaller size is more desirable for neurological and myocardial mapping/ablation leads and neuromodulation and stimulation leads.

The conductors may take the form of solid conductors, drawn-filled-tube (DFT), drawn-brazed-strand (DBS), stranded conductors or cables, ribbons conductors, or other forms known or recognized to those skilled in the art. The composition of the conductors may include aluminum, stainless steel, MP35N, platinum, gold, silver, copper, vanadium, alloys, or other conductive materials or metals known to those of ordinary skill in the art. The number, size, cross-sectional shape, and composition of the conductors will depend on the particular application for the lead body 120.

As previously mentioned, the conductors 420 may be wound along the first layer unitary body assembly 400 in a first direction (either a clockwise direction or a counterclockwise direction) around the lumen 450 at the center of the first layer unitary body assembly 400. If the clockwise direction is chosen as a first direction, then a second opposite direction is the counterclockwise direction. If the counterclockwise direction is chosen as a first direction, then a second opposite direction is the clockwise direction. The conductors 420 are typically insulated from the lumen 450, and from each other, and from the external surface of the first layer unitary body assembly 400 by the extrusion material. As also previously mentioned, the extrusion material may be of single composition, or of multiple layers of the same or different materials.

First layer unitary body assembly 400 is then covered with heat shrink tubing (not shown) and heat is applied. The heat melts the layers (410, 430 and 440) of extrusion material and the melted extrusion material flows together to form an integral body. The heat shrink tubing holds and compresses the extrusion material and the conductors that are located within the extrusion material to create a first layer unitary body 500 as shown in FIG. 5. The conductors 420 in first layer unitary body 500 may each be centered within the wall 510 of the first layer unitary body 500. Wall 510 is formed from materials that included the layers (410, 430 and 440) of extrusion material shown in FIG. 4. The first layer unitary body 500 is cooled and the heat shrink tubing removed. Lumen 520 is formed when the first layer unitary body 500 is removed from the mandrel (not shown) . There may be some release of coiled tension in the conductors 420 when the heat shrink tubing is removed.

The present invention provides a layer 430 of extrusion material around each conductor 420. This protective layer 430 of extrusion material provides an electrical barrier between each of the conductors 420. This provides a significant improvement over the prior art method that uses a mechanical comb in the winders to try to keep the conductors 420 separate. The protective layer 430 of extrusion material also allows the present invention to create leads that are smaller and thinner than prior art leads.

The method of the present invention provides several advantages over prior art methods. Advantages of the method of the present invention include: (1) more accurate conductor placement during the process of coiling the conductor around a mandrel, (2) more accurate conductor pitches, (3) improved pitch consistency, (4) more conductor protection during the process of coiling the conductor around the mandrel, and (5) precise centering of the conductors within the resulting unitary body.

Importantly, the apparatus and method of the present invention makes possible the construction of lead bodies that have a smaller diameter than prior art lead bodies. That is, the lead bodies of the present invention may be made thinner than prior art lead bodies. The cylindrically symmetrical embodiment of the lead body 120 of the invention can also better withstand lateral stretching than prior art lead bodies.

The first layer unitary body assembly 400 has been described as having cylindrical symmetry. It is noted that other types of geometrical cross-sectional shapes (e.g., rectangular) could be used if a different shape is desired for a particular application.

The first layer unitary body assembly 400 of lead body 120 has been shown as having four conductors 420. The use of four conductors 420 is merely an example. It is understood that more than four conductors 420 and fewer than four conductors 420 may be used. In one advantageous embodiment eight conductors 420 are used in the first layer unitary body assembly 400.

The method for forming first layer unitary body 500 of lead body 120 that has been described is not the only method that may be used. Other methods for forming first layer unitary body 500 are described in co-pending U.S. patent application Ser. No. [Attorney Docket No. 03-002], and are incorporated herein by reference for all purposes as if fully set forth herein.

After the first layer unitary body 500 of lead body 120 has been formed, additional conductors and extrusion material are applied over first layer unitary body 500 to form a second layer of lead body 120.

The above embodiment of the invention is generally described in U.S. patent application Ser. No. [Attorney Docket Number 03-003] filed concurrently herewith, entitled “System and Method for Providing A Medical Lead Body Having Dual Conductor Layers” without the inventive step of winding each layer in a direction counter to the other layer. Each of the embodiments as described in U.S. patent application Ser. No. [Attorney Docket Number 03-003] describes various other embodiments of the forming unitary lead bodies wherein the lead bodies, post manufacture, comprise a unitary wall with the conductors within the wall without the inventive step of winding each layer in a direction counter to the other layer. These descriptions are incorporated by reference here in full.

FIGS. 6-15 of this application illustrate these various embodiments. However, rather than as described in U.S. patent application Ser. No. [Attorney Docket Number 03-003], as they relate to the embodiments disclosed here, each first layer is spirally wound around the lumen or mandrel in a direction counter to or opposite to the second layer.

In other words, as previously mentioned, the conductors (such as 620 in FIG. 6) may be wound along the first layer unitary body assembly 600 in a first direction (either a clockwise direction or a counterclockwise direction) around the lumen 650 at the center of the first layer unitary body assembly 600. If the clockwise direction is chosen as a first direction, then a second opposite direction is the counterclockwise direction. If the counterclockwise direction is chosen as a first direction, then a second opposite direction is the clockwise direction. The conductors 620 are typically insulated from the lumen 650, and from each other, and from the external surface of the first layer unitary body assembly 600 by the extrusion material. As also previously mentioned, the extrusion material may be of single composition, or of multiple layers of the same or different materials. This counter rotational direction of each layer's spiral winding holds true for FIGS. 6-15.

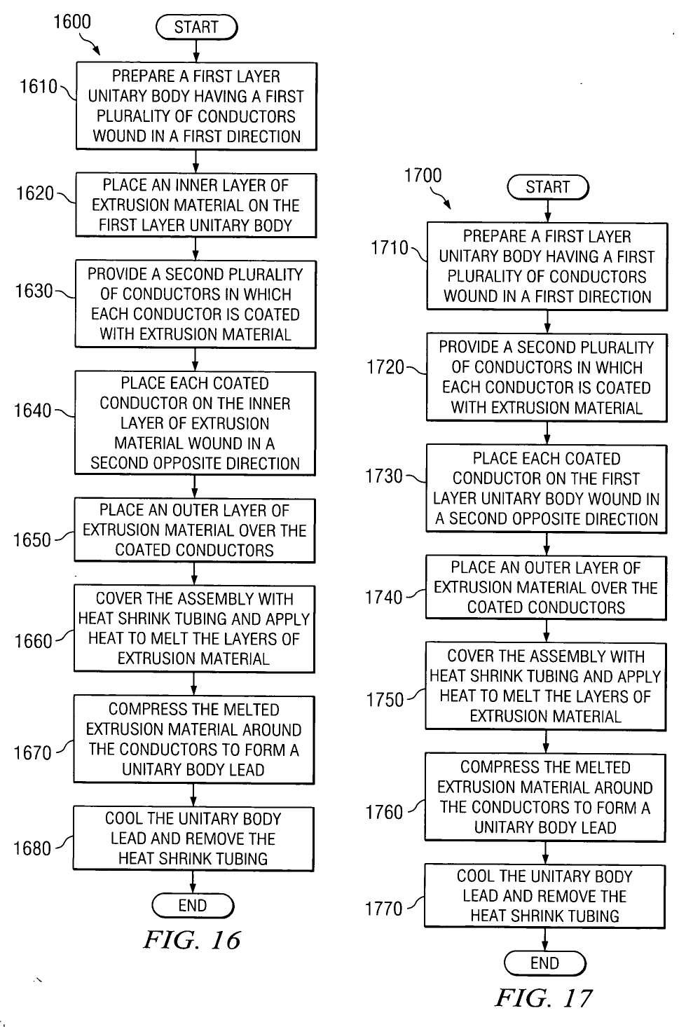

FIG. 16 illustrates a flow chart depicting the steps of one advantageous embodiment of the process of the present invention for making a first embodiment of lead body 120. The steps of the method are collectively referred to with reference numeral 1600.

A first body unitary layer 500 is prepared having a first plurality of conductors 420 wound in a first direction (step 1610). An inner layer of extrusion material is placed over the first layer unitary body 500 (step 1620). A second plurality of conductors is provided in which each conductor is coated with extrusion material (step 1630). Each coated conductor is then wound around the inner layer of extrusion material in a second opposite direction (step 1640). An outer layer of extrusion material is then placed over the second plurality of coated conductors on the inner layer (step 1650).

The assembly of the first layer unitary body, the inner layer, the coated conductors, and the outer layer is then covered with heat shrink tubing and heat is applied to melt the layers of extrusion material (step 1660). The heat shrink tubing compresses the extrusion material around the conductors to form a unitary body lead (step 1670). The unitary body lead is then cooled and the heat shrink tubing is removed (step 1680).

The methodologies described in U.S. patent application Ser. No. [Attorney Docket Number 03-003], which are incorporated by reference here in full, remain unchanged for the purpose of this invention, with the added feature that in the each of the steps of such figures that are concurrent, like or similar to steps 1610 and 1640 (in FIG. 16) has the added component of having the first layer of each embodiment spirally wound in a direction (for a step similar to step 1610), which is opposite to the spiral winding of the second layer (for a step similar to step 1640).

The methodologies described in U.S. patent application Ser. No. [Attorney Docket Number 03-003] that relate to FIGS. 21-24 are also incorporated by reference here in full.

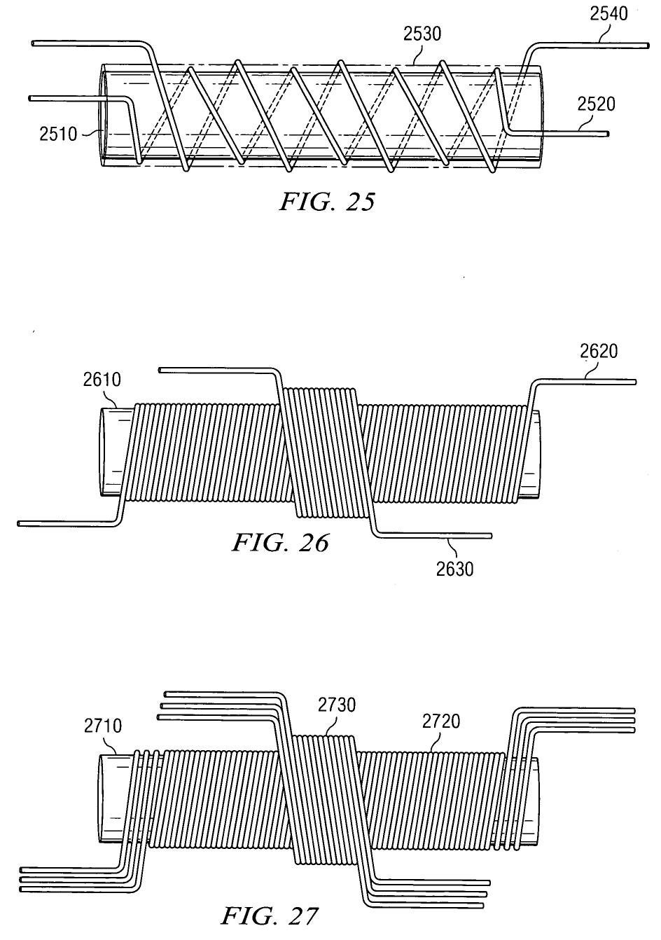

FIG. 25 illustrates a perspective side view of an exemplary mandrel 2510. FIG. 25 illustrates how an exemplary conductor 2520 of a first plurality of conductors may be wound around the axial length of the mandrel 2510 in a first direction within an inner layer of conductors. A cylinder 2530 is shown in dotted outline around mandrel 2510. Cylinder 2530 represents a boundary between an inner layer of conductors (e.g., first layer unitary body 500 or a non-unitary first layer) and an outer layer of conductors (wherein the outer layer may or may not be a unitary body). For clarity, the outer boundary of the outer layer of conductors is not shown in FIG. 25. An exemplary conductor 2540 of a second plurality of conductors may be wound around the axial length of mandrel 2510 within the outer layer of conductors. Exemplary conductor 2540 is wound in a second opposite direction with respect to conductor 2520. If conductor 2520 is wound in a clockwise direction, then conductor 2540 is wound in a counterclockwise direction. If conductor 2520 is wound in a counterclockwise direction, then conductor 2540 is wound in a clockwise direction. Electrical current in conductor 2540 may flow in the same direction or in the opposite direction as the electrical current in conductor 2520.

FIG. 26 illustrates a perspective side view of a portion of an exemplary mandrel 2610. FIG. 26 illustrates how a first conductor 2620 may be wound around the axial length of mandrel 2610 in a first direction within an inner layer of a lead body. FIG. 26 also illustrates how a second conductor 2630 may be wound around first conductor 2620 in an outer layer of the lead body along the axial length of the mandrel 2610. Second conductor 2630 is wound around first conductor 2630 in a second direction that is opposite to the direction of first conductor 2620.

FIG. 27 illustrates a perspective side view of a mandrel 2710 with an exemplary first plurality of conductors 2720 wound around the mandrel 2710 in a first direction in an inner layer of conductors and an exemplary second plurality of conductors 2730 wound around the mandrel 2710 in a second layer in a second opposite direction in a second layer of conductors.

It may be advantageous to set forth definitions of certain words and phrases that may be used within this patent document: the terms “include” and “include,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

1. A method for manufacturing a lead comprising the steps of:
placing on a mandrel, a first layer comprising at least one conductor of a first plurality of conductors wherein the at least one conductor of the first plurality of conductors is spirally wound in a first direction;
placing a second layer comprising at least one conductor of a second plurality of conductors on said first layer, wherein the at least one conductor of the second plurality of conductors is spirally wound in a second direction;
forming a lead body assembly that comprises the first layer and the second layer;
attaching at least one electrode located at a distal end of said lead body assembly to said at least one conductor of said first plurality of conductors;
attaching at least one connector located at a proximal end of said lead body assembly to said at least one conductor of said first plurality of conductors;
attaching at least one electrode located at a distal end of said lead body assembly to said at least one conductor of said second plurality of conductors;
attaching at least one connector located at a proximal end of said lead body assembly to said at least one conductor of said second plurality of conductors; and
removing the lead body assembly from the mandrel.

2. The method as claimed in claim 1 wherein the second direction is spirally wound in a direction opposite to the first direction.

3. The method as claimed in claim 2 wherein the first layer is a first unitary body.

4. The method as claimed in claim 2 wherein the second layer is a second unitary body.

5. The method as claimed in claim 1 wherein one of the first layer and the second layer further comprises extrusion material.

6. The method as claimed in claim 1 further comprising the step of forming the lead body assembly into a unitary lead body assembly.

7. The method as claimed in claim 6 wherein the step of forming the lead body assembly into a unitary lead body assembly further comprises the step of forming the first layer and the second layer into a unitary wall, wherein the at least one conductor of a first plurality of conductors and the at least one conductor of a second plurality of conductors are within the unitary wall.

8. The method as claimed in claim 1 wherein the first layer and the second layer further comprise extrusion material.

9. The method as claimed in claim 6 wherein the first layer and the second layer are comprised of similar types of extrusion material.

10. The method as claimed in claim 5 further comprising the steps of:
placing heat shrink tubing over the lead body assembly;
heating the lead body assembly to melt the extrusion material in the lead body assembly;
compressing the melted extrusion material around the at least one conductor of the second plurality of conductors in the lead body assembly;
cooling the lead body assembly to form the lead body; and
removing the heat shrink tubing from the lead body.

11. A lead for implantation into a human body, the lead comprising:
a lead body assembly comprising:
a wall having an inner portion that forms a lumen;
a first layer having at least one conductor spirally wound around the lumen in a first direction; and
a second layer having at least one conductor spirally wound around the lumen in a second direction and interior to the outside of the wall;
at least one electrode located at a distal end of said lead connected to said at least one conductor of said first plurality of conductors;
at least one electrode located at a distal end of said lead connected to said at least one conductor of said second plurality of conductors;
at least one connector located at a proximal end of said lead connected to said at least one conductor of said first plurality of conductors; and
at least one connector located at a proximal end of said lead connected to said at least one conductor of said second plurality of conductors.

12. The lead as claimed in claim 11 wherein the second direction is in a direction opposite to the first direction.

13. The lead as claimed in claim 12 wherein the first layer is a first unitary body.

14. The lead as claimed in claim 12 wherein the second layer is a second unitary body.

15. The lead as claimed in claim 11 wherein one of the first layer and the second layer further comprises extrusion material.

16. The lead as claimed in claim 11 wherein the lead body assembly is a unitary lead body assembly.

17. The lead as claimed in claim 11 wherein the wall is a unitary wall.

18. The lead as claimed in claim 17 wherein the unitary wall comprises of the first layer and the second layer, wherein the at least one conductor of a first plurality of conductors and the at least one conductor of a second plurality of conductors are within the unitary wall.

19. The lead as claimed in claim 11 wherein one of the first layer and the second layer further comprises extrusion material.

20. The lead as claimed in claim 19 wherein one of the first layer and the second layer are comprised of similar types of extrusion material.

21. The lead as claimed in claim 11, wherein the diameter of the lead is no greater than 34 French.

22. The lead as claimed in claim 11, further comprising at least five electrodes.

23. A system for stimulating a portion of a body, wherein the system comprises:
a source for generating a stimulus; and
a lead for receiving the stimulus from the source, wherein the lead comprises:
a lead body assembly comprising:
a wall having an inner portion that forms a lumen;
an first layer having at least one conductor spirally wound around the lumen in a first direction; and
an second layer having at least one conductor spirally wound around the lumen in a second direction and interior to the outside of the wall;
at least one electrode located at a distal end of the lead body; and
at least one connector located at a proximal end of the lead body, wherein the at least one connector and the at least one electrode are connected by at least one of the conductors.

24. The system as claimed in claim 23 wherein the second direction is in a direction opposite to the first direction.

25. The system as claimed in claim 24 wherein the first layer is a first unitary body.

26. The system as claimed in claim 24 wherein the second layer is a second unitary body.

27. The system as claimed in claim 23 wherein one of the first layer and the second layer further comprises extrusion material.

28. The system as claimed in claim 23 wherein the lead body assembly is a unitary lead body assembly.

29. The system as claimed in claim 23 wherein the wall is a unitary wall.

30. The system as claimed in claim 29 wherein the unitary wall comprises of the first layer and the second layer, wherein the at least one conductor of a first plurality of conductors and the at least one conductor of a second plurality of conductors are within the unitary wall.

31. The system as claimed in claim 30 wherein one of the first layer and the second layer further comprises extrusion material.

32. The system as claimed in claim 23 wherein the diameter of the lead is no greater than 34 French.

33. The system as claimed in claim 23 further comprising at least five electrodes.

34. A method for manufacturing a lead body comprising the steps of:
preparing a first layer unitary body comprising a first plurality of conductors;
placing at least one conductor of a second plurality of conductors on said first layer unitary body;
placing extrusion material over the at least one conductor of the second plurality of conductors to form a lead body assembly;
wherein each conductor in said first plurality of conductors in said first layer unitary body is wound in a first direction; and
wherein said at least one conductor of said second plurality of conductors is wound in a second opposite direction.

35. The method as claimed in claim 34 further comprising the steps of:
placing heat shrink tubing over the lead body assembly;
heating the lead body assembly to melt the extrusion material in the lead body assembly;
compressing the melted extrusion material around the at least one conductor of the second plurality of conductors in the lead body assembly;
cooling the lead body assembly to form the lead body; and
removing the heat shrink tubing from the lead body.

36. A method for manufacturing a lead body comprising the steps of:
preparing a first layer unitary body comprising a first plurality of conductors;
placing at least one conductor of a second plurality of conductors coated with a layer of extrusion material on the first layer unitary body;
wherein each conductor of said first plurality of conductors in said first layer unitary body is wound in a first direction; and
wherein said at least one conductor of said second plurality of conductors is wound in a second opposite direction.

37. A lead body assembly comprising:
a first layer unitary body comprising a first plurality of conductors;
an inner layer of extrusion material one the first layer unitary body;
a second plurality of conductors wherein each conductor of the second plurality of conductors is coated with a layer of extrusion material and wherein each conductor of the second plurality of conductors is placed on the inner layer of extrusion material;
wherein each conductor of said first plurality of conductors in said first layer unitary body is wound in a first direction; and
wherein each conductor of said second plurality of conductors is wound in a second opposite direction.

38. A system for stimulating a portion of a body, wherein the system comprises:
a source for generating a stimulus; and
a lead for receiving the stimulus from the source, wherein the lead comprises a lead body formed from a lead body assembly comprising:
a first layer unitary body comprising a first plurality of conductors;
an inner layer of extrusion material on the first unitary body;
a second plurality of conductors wherein each conductor of the second plurality of conductors is coated with a layer of extrusion material and wherein each conductor of the second plurality of conductors is placed on the inner layer of extrusion material; and
an outer layer of extrusion material placed over the second plurality of conductors;
wherein each conductor of said first plurality of conductors in said first layer unitary body is wound in a first direction; and
wherein each conductor of said second plurality of conductors is wound in a second opposite direction.

39. A method of manufacturing a lead body comprising the steps of:
placing on a mandrel a first layer unitary body comprising a first plurality of conductors; and
placing at least one conductor of a second plurality of conductors coated with a layer of extrusion material on said first layer unitary body to form a lead body assembly;
wherein each conductor of said first plurality of conductors in said first layer unitary body is wound in a first direction; and
wherein said at least one conductor of said second plurality of conductors is wound in a second opposite direction.

40. A lead body comprising:
a first layer unitary body comprising a first plurality of conductors; and
a second plurality of conductors in which each conductor of the second plurality of conductors is coated with a layer of extrusion material
wherein each conductor of said first plurality of conductors in said first layer unitary body is wound in a first direction; and
wherein each conductor of said second plurality of conductors is wound in a second opposite direction.
